(19) United States
(12) Patent Application Publication
(10) Pub. No.: US 2016/0331742 A1
(43) Pub. Date: Nov. 17, 2016

(54) TREATMENT OF CROHN'S DISEASE USING LOW DOSES OF LAQUINIMOD

(71) Applicants: Kurt A Brown, Narberth, PA (US); Ella Sorani, Kadima (IL)

(72) Inventors: Kurt A Brown, Narberth, PA (US); Ella Sorani, Kadima (IL)

(73) Assignee: Teva Pharmaceutical Industries, Ltd., Petach-Tikva (IL)

(21) Appl. No.: 15/112,101

(22) PCT Filed: Jan. 15, 2015
(86) PCT No.: PCT/US15/11587

§ 371 Date: Jul. 15, 2016

Related U.S. Application Data
(60) Provisional application No. 61/928,995, filed on Jan. 17, 2014.

(51) Int. Cl. A61K 31/4704; A61K 9/48

(52) U.S. Cl. CPC A61K 31/4704; A61K 9/48

(57) ABSTRACT

This invention provides a method of treating a subject suffering from Crohn's disease, comprising periodically administering to the subject an amount of laquinimod or pharmaceutically acceptable salt thereof effective to treat the subject, which amount of laquinimod is less than 0.5 mg/day, wherein the subject is naïve to laquinimod and the administration continues for at least 12 weeks, or wherein the subject is being treated with another Crohn's disease therapy at baseline. Furthermore, this invention provides a corresponding therapeutic package, use of laquinimod or pharmaceutically acceptable salt thereof in the manufacture of a medicament, and pharmaceutical composition.

This application claims benefit of U.S. Provisional Application No. 61/928,995, filed Jan. 17, 2014, the entire content of which is hereby incorporated by reference herein.

Throughout this application various publications are referenced. The disclosures of these publications in their entireties are hereby incorporated by reference into this application in order to mare fully describe the state of the art to which this invention pertains.

### BACKGROUND

Crohn's disease (GD) and Ulcerative Colitis (UC) are the two major types of Inflammatory Bowel Disease (IBD)—a generic classification for a group of nonspecific, idiopathic inflammatory disorders of the gastrointestinal (GI) tract which also includes Indeterminate Colitis (IC). Indeterminate Colitis refers to the up to 15% of IUD cases where distinguishing between CD and UC is impossible (Kasper, 2008). Both CD and UC tend to be chronic in nature and run a course characterized by exacerbations and remissions (US 2013/0203807).

CD may occur in any part of the GI tract, but most commonly affects the distal ileum and colon. It is characterized by transmural inflammation of the gastrointestinal wall, interspersed with “skip” areas of normal tissue, leading to the characteristic endoscopic and radiographic appearance of the disease. In about half the cases, biopsy specimens reveal the pathognomonic histology of noncaseating granulomas (Friedman, 2001).

The characteristic inflammatory presentation of Crohn's disease is of abdominal pain, diarrhea, fever and weight loss which may be complicated by intestinal fistulisation, obstruction, or both. Fistula formation may occur to the adjacent bowel, the skin, the urinary bladder, or other locations. Obstruction, if present, is initially intermittent due to bowel wall edema and spasm; further progression may lead to chronic scarring and stricture formation. Perianal disease is common and may manifest as anal fissure, perianal fistula, or abscess (Friedman, 2001; Wu, 2007).

Extra-intestinal manifestations may also occur and include joint inflammation (e.g., peripheral arthritis, ankylosing spondylitis), skin lesions (e.g., erythema nodosum, pyoderma gangrenosum), ocular involvement (e.g., iritis, uveitis) and liver disorders (e.g., hepatic steatosis, primary sclerosing cholanitis) (Friedman, 2001).

The incidence of CD varies within different geographic areas. Northern countries such as the US, UN, Norway and Sweden have the highest rates. The incidence of CD in the US is approximately 7 per 100,000. Countries in southern Europe, South Africa and Australia have lower incidence rates of 0.9 to 3.1 per 100,000. The disease is rare in Asia and South America (Friedman, 2001).

The peak age of onset of Crohn's disease occurs between the ages of and 30 years, with a second peak of occurrence between the ages of 60-80 years (Friedman, 2001).

The fundamental cause of CD is unknown. There are four basic factors affecting the pathophysiology of CD genetics, immune dysregulation, epithelial barrier dysfunction and the constitution of microbial flora. Evidence suggests that genetic predisposition leads to an unregulated intestinal immune response to an environmental, dietary or infectious agent (Friedman, 2001; Wan, 2004), A number of studies suggest that CD is a T-helper 1 (Th-1) mediated disease and that the excessive Th1-cell activity leading to the production of a wide range of proinflammatory cytokines [including interleukin (IL)-1, IL-2 and tumor necrosis factor (TNF)-α] and an imbalance between proinflammatory and anti-inflammatory reactivity, is a critical component of CD (Hendrickson, 2002). However, no inciting antigen has been identified.

In the absence of a key diagnostic test, the diagnosis of Crohn's disease is based on endoscopic, radiographic and pathological findings documenting focal, asymmetric transmural or granulomatous features. Laboratory abnormalities include non-specific markers of inflammation such as elevated sedimentation rate and C-reactive protein (CRP). In more severe cases, finding may include hypoalbuminemia, anemia, and leukkocytosis (Friedman, 2001; Wu, 2007).

The major therapeutic goals in CD treatment are the reduction of signs and symptoms, induction and maintenance of remission and most importantly, the prevention of disease progression and complications.

Sulfasalazine and other 5-aminesalicylic acid agents, antibiotics such as metronidazole and ciprofloxacin, corticosteroids, immunosupressors such as azathioprine and 6-mercaptopurine and biologic agents such as anti-TNFα agents and anti-integrins that prevent leukocyte infiltration have shown to be useful in the induction of remission and/or in its maintenance (Targan, 1977; Hanauer, 2002; Colombel, 2007; Ghosh, 2003; Sandbozn, 2005; Schreiber, 2005; Schreiber, 2007; Kozuch, 2008). Many of these medicinal products, however, are only moderately efficacious and are associated with challenging side effects (Homes, 2003; Thomas, 2004; Colombel, 2004; Van Assche, 2005; Vermeire, 2003; Sweetman, 2006). In addition, the newer biologic agents have a relatively inconvenient parenteral route of administration.

More recently, laquinimod has been suggested for use in treating a subject suffering from Crohn's disease, wherein the dose is 0.5 mg/day laquinimod, as described in U.S. patent Pub. No. 2011/0027219. Laquinimod is a novel synthetic compound with high oral bioavailability, which has been suggested as an oral formulation for Relapsing Remitting Multiple Sclerosis (MS). Laquinimod and its sodium salt form are described, for example, in U.S. Pat. No. 6,077,851.

Disclosed herein is a method of treating a subject suffering from Crohn's disease using laquinimod, where the method comprises periodically administering to the subject an amount of laquinimod or pharmaceutically acceptable salt thereof effective to treat the subject, which amount of laquinimod is less than 0.5 mg/day.

### SUMMARY OF THE INVENTION

The subject invention provides a method of treating a subject suffering from Crohn's disease, the method comprising of periodically administering to the subject an amount of laquinimod or pharmaceutically acceptable salt thereof effective to treat the subject, which amount of laquinimod is less than 0.5 mg/day, wherein the subject is naïve to laquinimod and wherein the administration continues for at least 12 weeks.

The subject invention also provides a method of treating a subject suffering from Crohn's disease, the method comprising of periodically administering to the subject an amount of laquinimod or pharmaceutically acceptable salt thereof effective to treat the subject, which amount of laquinimod is less than 0.5 mg/day and wherein the subject is being treated with another Crohn's disease therapy at baseline.

The subject invention also provides laquinimod for use in treating a subject suffering from Crohn's disease, wherein the amount of laquinimod is less than 0.5 mg/day, wherein the subject is naïve to laquinimod and wherein the administration continues for at least 12 weeks.

The subject invention also provides laquinimod for use in treating a subject suffering from Crohn's disease, wherein the amount of laquinimod is less than 0.5 mg/day, wherein the subject is being treated with another Crohn's disease therapy at baseline.

The subject invention also provides a pharmaceutical composition comprising an amount of laquinimod for use in treating a subject suffering from Crohn's disease, wherein the amount of laquinimod is less than 0.5 mg, wherein the subject is naïve to laquinimod and wherein the administration continues for at least 12 weeks.

The subject invention also provides a pharmaceutical composition comprising an amount of laquinimod for use in treating a subject suffering from Crohn's disease, wherein the amount of laquinimod is less than 0.5 mg, wherein the subject is being treated with another Crohn's disease therapy at baseline.

The subject invention also provides use of an amount of laquinimod or a pharmaceutically acceptable salt thereof in the manufacture of a medicament for the treatment of a subject suffering from Crohn's disease, wherein the amount of laquinimod is less than 0.5 mg, wherein the subject is nave to laquinimod and wherein the administration continues for at least 12 weeks.

The subject invention also provides use of an amount of laquinimod or a pharmaceutically acceptable salt thereof in the manufacture of a medicament for the treatment of a subject suffering from Crohn's disease, wherein the amount of laquinimod is less than 0.5 mg, wherein the subject is being treated with another Crohn's disease therapy at baseline.

The subject invention also provides a therapeutic package for dispensing to, or for use in dispensing to, a subject suffering from Crohn's disease and naïve to laquinimod, which comprises:
a) one or more unit doses, each such unit dose comprising an amount of laquinimod, and
b) a finished pharmaceutical container therefor, said container containing said unit dose or unit doses, said container further containing or comprising labeling directing the use of said package in the treatment of said subject for a period of at least 12 weeks.

The subject invention also provides a therapeutic package for dispensing to, or for use in dispensing to, a subject suffering from Crohn's disease and being treated with another Crohn's disease therapy at baseline, which comprises:
a) one or more unit doses, each such unit dose comprising an amount of laquinimod, and
b) a finished pharmaceutical container therefor, said container containing said unit dose or unit doses, said container further containing or comprising labeling directing the use of said package in the treatment of said subject.

### DETAILED DESCRIPTION OF THE INVENTION

This application provides for a method of treating a subject suffering from Crohn's disease, the method comprising of periodically administering to the subject an amount of laquinimod or pharmaceutically acceptable salt thereof effective to treat the subject, which amount of laquinimod is less than 0.5 mg/day, wherein the subject is naïve to laquinimod and wherein the administration continues for at least 12 weeks.

This application also provides for a method of treating a subject suffering from Crohn's disease, the method comprising of periodically administering to the subject an amount of laquinimod or pharmaceutically acceptable salt thereof effective to treat the subject, which amount of laquinimod is less than 0.5 mg/day and wherein the subject is being treated with another Crohn's disease therapy at baseline.

In one embodiment, the amount of laquinimod is 0.25 mg/day.

In another embodiment, the amount of laquinimod is effective to reduce a symptom of Crohn's disease in the subject.

In another embodiment, the amount of laquinimod is effective to induce or maintain clinical remission in the subject.

In another embodiment, the amount of laquinimod is effective to induce C-Reactive Protein response.

In another embodiment, the amount of laquinimod is effective to reduce an endoscopic disease activity in the subject.

In another embodiment, the amount of laquinimod is effective to induce CDEIS response.

In another embodiment, the amount of laquinimod is effective to induce CDEIS improvement.

In another embodiment, the amount of laquinimod is effective to induce CDEIS remission.

In another embodiment, the amount of laquinimod is effective to induce mucosal healing.

In one embodiment, the endoscopic disease activity is measured by the Crohn's Disease Endoscopic Index of Severity CDEIS).

In another embodiment, the endoscopic disease activity is measured by the Simple Endoscopic Score for Crohn's Disease (SES-CD).

This application also provides for laquinimod for use in treating a subject suffering from Crohn's disease, wherein the amount of laquinimod is less than 0.5 mg/day, wherein the subject is naïve to laquinimod and wherein the administration continues for at least 12 weeks.

This application also provides for laquinimod for use in treating a subject suffering from Crohn's disease, wherein the amount of laquinimod is less than 0.5 mg/day, wherein the subject is being treated with another Crohn's disease therapy at baseline.

This application also provides for a pharmaceutical composition comprising an amount of laquinimod for use in treating a subject suffering from Crohn's disease, wherein the amount of laquinimod is less than 0.5 mg, wherein the subject is naïve to laquinimod and wherein the administration continues for at least 12 weeks.

This application also provides for a pharmaceutical composition comprising an amount of laquinimod for use in treating a subject suffering from Crohn's disease, wherein the amount of laquinimod is less than 0.5 mg, wherein the subject is being treated with another Crohn's disease therapy at baseline.

This application also provides for use of an amount of laquinimod or a pharmaceutically acceptable salt thereof in the manufacture of a medicament for the treatment of a subject suffering from Crohn's disease, wherein the amount of laquinimod is less than 0.5 mg, wherein the subject is naïve to laquinimod and wherein the administration continues far at least 12 weeks.

This application also provides for use of an amount of laquinimod or a pharmaceutically acceptable salt thereof in the manufacture of a medicament for the treatment of a subject suffering from Crohn's disease, wherein the amount of laquinimod is less than 0.5 mg, wherein the subject is being treated with another Crohn's disease therapy at baseline.

This application also provides for a therapeutic package for dispensing to, or for use in dispensing to, a subject suffering from Crohn's disease and naïve to laquinimod, which comprises:
a) one or more unit doses, each such unit dose comprising an amount of laquinimod, and
b) a finished pharmaceutical container therefor, said container containing said unit dose or unit doses, said container further containing or comprising labeling directing the use of said package in the treatment of said subject for a period of at least 12 weeks.

This application also provides for a therapeutic package for dispensing to, or for use in dispensing to, a subject suffering from Crohn's disease and being treated with another Crohn's disease therapy at baseline, which comprises:
a) one or more unit doses, each such unit dose comprising an amount of laquinimod, and
b) a finished pharmaceutical container therefor, said container containing said unit dose or unit doses, said container further containing or comprising labeling directing the use of said package in the treatment of said subject.

For the foregoing embodiments, each embodiment disclosed herein is contemplated as being applicable to each of the other disclosed embodiments. For instance, the elements recited in the method embodiments can be used in the pharmaceutical composition, package, product and use embodiments described herein and vice versa.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/04899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit may be in a form suitable for oral administration Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agentS, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,509,208, PCT International Application Publication Nos. WO 2005/074899, WO 2007/047863, and 2007/146248.

General techniques and compositions for making dosage forms useful in the present invention are described-in the following references:

Modern Pharmaceutics, Chapters 9 and 10 (Banker & Rhodes, Editors, 1979); Pharmaceutical Dosage Forms: Tablets (Lieberman et al., 1.961); Ansel, Introduction to pharmaceutical Dosage Forms 2nd Edition (1976); Remington's Pharmaceutical Sciences, 17th ed. (Mack Publishing Company, Easton, Pa., 1965); Advances in Pharmaceutical Sciences (David Ganderton, Trevor Jones, Eds., 1992); Advances in Pharmaceutical Sciences Vol 7. (David. Ganderton, Trevor Jones, James McGinity, Eds, 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds); Modern Pharmaceutics Drugs and the Pharmaceutical sciences, Vol. 40 (Gilbert S. Banker, Christopher T. Rhodes, Eds). These references in their entireties are hereby incorporated by reference into this application.

Disclosed is a method for treating a subject suffering from Crohn's disease comprising of periodic administration of an amount of laquinimod effective to treat the subject, which amount of laquinimod is less than 0.5 mg/day, wherein the subject is naïve to laquinimod and wherein the administration continues for at least 12 weeks. Also disclosed is a method of treating a subject suffering from Crohn's disease comprising periodic administration of an amount of laquinimod effective to treat the subject, which amount of laquinimod is less than 05 mg/day and wherein the subject is being treated with another Crohn's disease therapy at baseline.

Terms

As used herein, and unless stated otherwise, each of the following terms shall have the definition set forth below.

As used herein, “laquinimod” means laquinimod acid or a pharmaceutically acceptable salt thereof.

As used herein “suffering”, as in a subject suffering from a disease or a condition, means a patient who has been clinically diagnosed to have the disease or condition. For example, “a subject suffering from Crohn's disease” means a subject who has been clinically diagnosed to have Crohn's disease. The diagnosis of the disease or condition can be affected using any of the appropriate methods known in the art. Thus, in an embodiment of the present invention the method includes the step of determining whether a patient is a Crohn's disease patient.

As used herein, a subject or a patient at “baseline” is a subject or patient prior to initiating periodic administration of laquinimod in a therapy as described herein.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

A “symptom” associated with a disease or disorder includes any clinical or laboratory manifestation associated with the disease, disorder or condition, e.g., a pathological condition and is not limited to what the subject can feel or observe.

An “amount” or “dose” of laquinimod as measured in milligrams refers to the milligrams of laquinimod acid present in a preparation, regardless of the form of the preparation.

As used herein, a “unit dose”, “unit doses” and “unit dosage form(s)” mean a single drug administration entity/entities.

As used herein, a “loading dose” refers to an initial higher dose of a drug that may be given at the beginning of a course of treatment before dropping down to a lower “intended dose” or “maintenance dose”.

As used herein, “about” in the context of a numerical value or range means±10% of the numerical value or range recited or claimed.

As used herein, “treating” encompasses, e.g., inducing inhibition, regression, or stasis of a disease or disorder, e.g., Crohn's disease, or alleviating, lessening, suppressing, inhibiting, reducing the severity of, eliminating or substantially eliminating, or ameliorating a symptom of the disease or disorder.

“Inhibition” of disease progression or disease complication in a subject means preventing or reducing the disease progression and/or disease complication in the subject.

As used herein, “effective” when referring to an amount of laquinimod refers to the quantity of a laquinimod that is sufficient to yield a desired therapeutic response without undue adverse side effects (such as toxicity, irritation, or allergic response) commensurate with a reasonable benefit/risk ratio when used in the manner of this invention.

As used herein, “Crohn's Disease Activity Index” or “CDAI” is a research tool developed by WR Best and colleagues from the Midwest Regional Health Center in Illinois, in 1976 (Best, 1976) to quantify the symptoms of patients with Crohn's disease. The index is the most widely used instrument for evaluation of Crohn's disease activity (Best, 1976; Best, 1979; Bandborn, 2002) and consists of eight factors/variables.

The eight variables are summed after adjustment with a weighting factor. The components of the COAT and weighting factors are shown in the following table 1:

TABLE 1

| | Weighting |
|---|---|
| Clinical or laboratory variable | factor |
| Number of liquid or soft stools (sum of each day for 7 days) | ×2 |
| Abdominal pain (graded from 0-3 on severity) (sum of each day for 7 days) | ×5 |
| General well being, subjectively assessed from 0 (well) to 4 (terrible) (sum of each | ×7 |
| day for 7 days) | |
| Presence of Crohn's disease complications | ×20 |
| Use of dyphenoxylate or loperamide for diarrhea during the past week (0 = no, 1 = | ×30 |
| yes) | |
| Presence of an abdominal mass (0 as none, 2 as questionable, 5 as definite) | ×10 |
| Absolute deviation of Hematocrit from 47% in men and 42% in women | ×6 |
| Percentage deviation from standard weight | ×1 |

The first 4 of these variables and the presence of fever above 37.6° C., are self-reported in subject diaries, the remaining 4 are assessed at the study visit. Height and standard weight assessment are based on standard height-weight tables.

Total CDAI scores range from 0 to approximately 600 where the higher the score, the more active the disease. A CDAI score of less than 150 points denotes “clinical remission” of the Crohn's disease, of between 150 to 219 points denotes “active mild Crohn's disease”, of between 220 to 450 points denotes “active moderate Crohn's disease” and of more than 450 points denotes “active severe Crohn's disease”.

“Clinical response” means that the subject's Crohn's disease symptoms have decreased in severity and/or in number. “Clinical remission” means that the subject's Crohn's disease symptoms have decreased in severity and/or in number to below a defined level, e.g., below 150 points on the CDAI scale. “Clinical remission” and “clinical response” may be measured in accordance with the EMEA draft guidelines on the development of new medicinal products for the treatment of Crohn's disease. The EMEA guidelines define “clinical remission” as reduction in CDAI score to a total score below 150 points and “clinical response” as if remission has been achieved or a reduction of at least 100 points in the total. CDAI score has been observed, compared to baseline at the end of the treatment period (EMEA, 2007).

“Indeterminate Colitis” or “IC” is used clinically in patients with some form of Inflammatory Bowel Disease in whom a definite diagnosis of either Ulcerative Colitis (UC) or Crohn's Disease (CD) has not been made, either on colonoscopy or colonic biopsy before colectomy. Although some patients diagnosed with Indeterminate Colitis go on to develop UC or CD, studies have shown that over a median follow up period of 10 years, many patients retain diagnosis of Indeterminate Colitis. (Guindi, 2004)

As used herein, “C-reactive protein” or “CRP” is an inflammatory mediator whose levels are raised under conditions of acute inflammatory recurrence and rapidly normalize once the inflammation subsides. Crohn's disease may be characterized according to disease behavior: predominantly nonstricturing nonpenetrating (inflammatory), stricturing or penetrating (Silverberg, 2005). The origin of symptoms such as diarrhea, fatigue, or abdominal pain (affects the CDAI score) may be multifactoral and does not necessarily correlate with the existence of prominent inflammatory lesions of the gastrointestinal (GU tract. Predominantly nonstricturing nonpenetrating (inflammatory) Crohn's disease may be characterized by high CRP levels. Therefore the CRP may serve as a surrogate marker to monitor inflammatory disease activity and response to treatment (Salem, 2005; Denis, 2007; Chamouard, 2006).

“C-reactive protein response” or “CRP response” have a varied definition based on the level at Baseline. For Baseline CRP level ≧10 mg/L, level needs to be reduced by at least 50% and to be <10 mg/L. For Baseline CRP level ≧5 mg/L and <10 mg/L, level needs to be reduced by at least 50% compared to Baseline OR to <5 mg/L. For Baseline CRP level <5 mg/L, level needs to remain <5 mg/L.

As used herein, “Crohn's Disease Endoscopic Index of Severity” or “CDEIS” is a score based on the presence, of deep or superficial ulcerations in the following segments: (1) rectum, (2) sigmoid and left colon, (3) transverse colon, (4) right colon, and (5) terminal ileum. In addition, the extent of the diseased and ulcerated areas are estimated in each segment, recorded by positioning a cross on two 10-cm linear analog scales, between 0 (no lesion or no ulceration at all) and 10 (lesions or ulcerations involving 100% of the segmental surface). These segmental data are recorded on a standard form, together with the endoscopist's global estimate of lesion severity. The presence of ulcerated or non-ulcerated stenosis is also factored into the score.

“Crohn's Disease Endoscopic Index of Severity Response” or “CDEIS Response” is a reduction of CDEIS by at least 50% compared to Baseline. “Crohn's Disease Endoscopic Index of Severity Improvement” or “CDEIS Improvement” is a CDEIS score <6. “Crohn's Disease Endoscopic Index of Severity Remission” or “CDEIS Remission” is a CDEIS score <3.

As used herein, “mucosal healing” is the total and/or complete absence of any endoscopic ulcer.

As used herein, “Simple Endoscopic Score; for Crohn's Disease” or “SES-CD” is a score that includes I variables: ulcer size, the extent of ulcerated surface, extent of affected surface, and stenosis, from 0 (no ulcers present) to 3 (large ulcers, >2 cm), in the same 5 segments of the bowel as in the CDEIS (Sipponen, 2010).

As used herein, “calprotectin” is a calcium and zinc binding anti-microbial protein released by granulocytes. This protein can be detected in stool and its concentration reflects the number of polymorphonuclear leukocytes (PMN), migrating into the gut lumen. It is therefore considered a bio-marker for intestinal inflammation.

“Adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product.

A “salt thereof” is a salt of the instant compounds which have been modified by making acid or base salts of the compounds. The term “pharmaceutically acceptable salt” in this respect, refers to the relatively non-toxic, inorganic and organic acid or base addition salts of compounds of the present invention. For example, one means of preparing such a salt is by treating a compound of the present invention with an inorganic base.

As used herein, “pharmaceutically acceptable carrier” refers to a carrier or excipient that is suitable for use with humans and/or animals without undue adverse side effects such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “5-10%” includes 5.0%, 5.1%, 5.2%, 5:3%, 5.4% etc. up to 10.0%.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Clinical Trial (Phase III)—Assessment of Oral Laquinimod in Active Moderate to Severe Crohn's Disease

A phase III, multicenter, randomized, double-blind, placebo-controlled study is conducted to evaluate Laquinimod in active moderate to severe Crohn's disease.

Study Title

A Phase Multicenter, Randomized, Double-Blind, Placebo-Controlled Study to Evaluate the Efficacy, Safety, Tolerability of Laquinimod in Active Moderate to Severe Crohn's Disease.

Number of Investigational Centers

There: are approximately 200 investigational centers.

Number of Subjects

There are ˜540 Crohn's disease patients enrolled, divided into 3 arms of ˜180 patients, randomized in a 1:111 ratio (˜180 subjects on laquinimod 0.25 mg, ˜180 on laquinimod 0.5 mg, and ˜180 on placebo).

Investigational Medicinal Product (IMP) & Dosage

One or more capsules containing laquinimod 0.25 mg, laquinimod 0.5 mg, or matching placebo are administered orally once daily;

1^{st} arm—laquinimod 0.25 mg (1×0.25) and placebo capsule for 01.5 mg (1× placebo capsule for 0.5 mg);

2^{nd} arm—laquinimod 0.5 mg (1×0.5) and placebo capsule for 0.25 rug (1× placebo capsule for 0.25 mg); and

3^{rd} arm—placebo capsule for 0.25 mg (1× placebo capsule for 0.25 mg) and placebo capsule for 0.5 mg (1× placebo capsule for 0.5 mg).

The 0.25 mg and 0.5 mg laquinimod capsules are packaged in high density polyethylene, 50-ml, (DUMA) bottles with 2 g of silica gel in cap, 35 capsules per bottle.

A loading dose regimen of double the maintenance/intended dose is given during the first two days of study drug treatment. Thereafter, starting on day 3, the daily maintenance/intended dose is administered.

For the 0.25 mg laquinimod dose group, on Days 1 and 2, this dose is administered twice daily (BID), approximately 12 hours apart, as a loading dose (total daily dose 0.5 mg), Dosing is once daily (0) from Day 3 onwards.

For the 0.5 mg laquinimod dose group, on Days 1 and 2, this dose is administered twice daily (BID), approximately 12 hours apart, as a loading; dose (total daily dose 1 mg). Dosing is once daily (QD) from Day 3 onwards.

For the placebo capsule dose group, an Days 1 and 2, this dose is administered twice daily (BID), approximately 12 hours apart, as a loading dose (total daily dose 0 mg). Dosing is once daily (CD) from Day 3 onwards.

Subjects are required to maintain CDAI diary cards for reporting daily symptoms from after the initial screening visit until the end of their participation in the study. The scores obtained from the seven consecutive diaries completed prior to each of weeks 2, 4, and 12 (and at Follow-up for patients who did not continue into a subsequent study) and after the initial Screening visit (Visit 1) contribute to a total CDAI score at each of the time points.

Allowed previous standard of care treatment is kept stable throughout the study (including the follow-up period, as defined herein).

Study Duration

Each Arm (Dose Group) is Evaluated for Up to 15.5 Weeks

Screening: up to 2.5 weeks

Treatment period: Up to 13 weeks

Follow-up period: 4 weeks

Study Population

Patients with moderate to severe Crohn's disease (CD), as determined by a Crohn's Disease Activity Index (CDM) score of 220-450 (inclusive) and evidence of active endoscopic inflammatory disease, based on Crohn's Disease Endoscopic index of Severity (CDEIS) score >6 points.

Study Design

This is a Phase III, randomized, double-blind, placebo-controlled study to assess the efficacy, safety, and tolerability of 2 doses of laquinimod in active moderate to severe CD patients.

This study investigates laquinimod doses at 0.25 and 0.5 mg daily.

Each dose is studied in a distinct arm.

Subjects are assessed for study eligibility up to 2.5 weeks prior to baseline.

Approximately 180 eligible subjects are assigned to each arm. Subjects were randomized in a 1:1:1 ratio for one of the following treatment arms:

1. Laquinimod 0.25 mg (˜180 subjects).

2. Laquinimod 0.5 mg 1˜180 subjects).

3. Placebo (˜180 subjects).

The laquinimod capsules (0.25 mg and 0.5 mg) are of identical appearance to their matching placebo (white opaque cap and body;

hard gelatin capsules filled with white to off-white granulate) to maintain study blinding.

Scheduled in-clinic visits are conducted at screening, baseline (week 0) and at weeks 2, 4, 8, and 12. The Week 12 visit is divided into 2 discrete visits, designated Week 12a (e.g., collection of most efficacy and safety data) and Week 12b (e.g., ileocolonoscopy). The Week 12b visit takes place within 5 days of the Week 12a visit.

Unscheduled visits for safety or for any other reason may be conducted at any time during the study.

During the study period the CDAI score is assessed in addition to routine safety laboratory tests and PK analysis.

A loading dose regimen of the study drug is given during the first two days of treatment (day 1/baseline and thereafter). The first loading dose of the study is administered at the site. The loading dose is double the intended dose for the first two days and is administered BID with 12 hour interval between dosing. Thereafter, starting on day 3, the dosing regimen consists of the intended dose once daily (QD):
1. Day 1 (Baseline): loading dose of the drug (intended dose at 0 hour, at the site and intended dose at 12 hours). Total dose was twice the intended dose.
2. Day 2: loading dose of the study drug (intended dose at 0 hour and intended dose at 12 hours). Total dose was twice the intended dose.
3. Day 3: Intended/maintenance dose of the study drug.

Patients are required to report daily symptoms diary data for the assessment of the CDAI, from the initial Screening visit until the end of their participation in the study. The scores obtained from the 7 consecutive days of CDAI data completed prior to each visit on Weeks 2, 4, 8, and 12 (and Fellow-up for patients who do not continue into a subsequent study) and after the initial Screening visit (Visit 1) contribute to a total CDAI score at each of these time points. Ileocolonoscopy is performed during Screening (Visit 2) and again at Week 12 (Week 12b). The timing of the ileocelonosoopy is arranged so that it takes place after the last day on which data are recorded for the CDAI assessment. At Screening, continued eligibility to participate in the study is based on the CDAI prior to the ileocolonoscopy procedure. Each endoscopy is recorded, and then read and scored by a central reader for endoscopic scores of CDEIS and SES-CD.

Assessments of abdominal pain (using the UPS) and stool frequency and consistency (using the 855) is performed daily throughout the study. Patients are required to complete the IBM, WPAI:CD, and EQ-SD questionnaires at Screenilg, Week 4, and Week 12 (and Follow-up for those who do not continue into a subsequent study),

Allowed previous standard of care treatment is kept stable throughout the study (including the follow-up period as defined herein).

PK Analysis

Population PK Study (PPK)

Blood samples for PPK evaluation are collected at weeks 4 and 8 from all subjects in all arms.

Pharmacogenetic Sub-Study

Blood samples for the pharmacogenetic sub-study were collected from all subjects who sign the separate informed consent form and upon Ethics Committee (EC) approval.

Alloyed Concomitant Medications During Study

In general the dose of allowed concomitant medication is kept stable throughout the study (including the follow-up period). Any new medication/treatment far CD or dose increase not allowed, by the protocol, throughout the study treatment period, results in major protocol violation and is regarded as a treatment failure. Decrease in dose or dose regiment, not allowed by the protocol, also results in major protocol violation.

CD surgery, biologic treatment or new immunosuppressive drugs/throughout the study treatment period, are regarded as treatment failure and results in early treatment discontinuation.

5-ASA Compounds

The use of 5-ASA compounds is kept stable throughout the study.

Antibiotics

The use of antibiotics for the treatment of Crohn's disease is kept stable throughout the trial. Managing acute infections (not related to Crohn's disease) is allowed. The use of antibiotics, such as ciprofloxacin, erythromycin, clarithromycin, troleandomycin and telithromycin which inhibit CYP 3A4, is not allowed.

Gluoticocosteroids

The dose of oral glucocorticosteroids remains stable throughout the study;
1. Oral systemic glucocorticosteroids—no more than prednisolone 2.5 mg/day (or equivalent) increase or decrease compared to baseline.
2, Budesonide—no change is permitted compared to baseline.
3. IV or IM corticosteroid dose or corticosteroid enemas are not allowed.

Immunosuppressants

Immunosuppressive treatment allowed by the protocol (AZT/6-MP/MTX) is kept stable throughout the study. Addition of new immunosuppressive drugs is not allowed.

Other
1. Antidiarrehal drugs, analgesics, NSAIDs and topical preparations are allowed. (including topical dermatological, opthalmological or inhaled steroids).
2. The use of probiotics and probiotics is kept stable throughout the study.

Inclusion/Exclusion Criteria

Inclusion Criteria

Subjects must meet all he inclusion criteria to be eligible:
1. Males and females 18-75 years old (inclusive).
2. Subjects diagnosed with Crohn's disease for at least 3 months prior to screening, which has been appropriately documented and supported by endoscopy, radiology, or surgery (performed within 36 months prior to screening).
3. Moderate to severe Crohn's disease patients as determined by a CCAI score of 220-450 (inclusive).
4. Evidence of active endoscopic inflammatory disease, based on CDEIS >6 points as measured by ileocolonoscopy.
5. Patients currently being treated with at least 1 medication intended for CD.
6. Subjects willing and able to provide written, informed consent.

Exclusion Criteria

Any of the following excludes the subject from entering the study:
1. Subjects with a diagnosis of indeterminate or ulcerative colitis.
2 Subjects with positive results on stool microbiology for enteric pathogens (Salmonella, Shigella, Yersinia, Campylobacter or Clostridia difficile toxin assay) at screening.
3. Subjects who have had bowel surgery or resection within the 3 months prior to screening.
4. Subjects with clinically significant short bowel syndrome or subtotal colectomy.
5. Subjects with clinically significant or symptomatic GI obstructive symptoms.
6. Subjects with intra-abdominal abscess or suspected abcess.
7. Subjects with ostomy, ileoanal pouch, or subtotal colectomy.
8. Subjects who receive parenteral nutrition.
9. Subjects undergoing oral corticosteroid treatment (e.g., prednisolone/prednisone/budesonide) initiated less than 4 weeks prior to screening.
10. Subjects being treated with prednisolone >20 mg/day (or equivalent) or budesonide >6 mg/day for CD at screening, or whose corticosteroid dosage regimen is not stable for at least 4 weeks prix to screening.
11, Subjects who receive intravenous or intramuscular corticosteroid administration within 2 weeks of screening.
12. Subjects being treated with 5-aminosalicylic acid that has not been at a stable dose for at least 2 weeks prior to screening.
13. Subjects being treated with antibiotics for CD that are not at a stable dose for at least 2 weeks prior to screening,
14. Subjects being treated with 6-mercaptpurine, azathioprine, or methotrexate that was initiated within 8 weeks prior to screening or has not been at a stable dose for at least 6 weeks prior to screening.
15. Subjects being treated with anti-tumor necrosis factor (TNF) within 4 weeks prior to screening.
16. Subjects being treated with cyclosporine, tacrolimus, mycophenolate mofetil, or thalidomide within 8 weeks prior to screening.
17. Subjects being treated with natalirumab/vedolizumab, ustekinumab within 8 weeks prior to screening.
16. Subjects with a clinically significant or unstable medical or surgical condition that, in the investigator's opinion, would preclude safe and complete study participation, as determined by medical history, physical examinations, ECG, or laboratory evaluations.
19. Subjects with any current diagnosis that could confound interpretation of safety (e.g., human immunodeficiency virus infection, hepatitis B or C) or efficacy (e.g., celiac disease, irritable bowel syndrome, bile salt diarrhea).
20. Subjects with planned or elective surgery or hospitalization during the course of the study that may interfere with study compliance or outcome.
21. Subjects with a upper limit of normal (ULN) of alanine aminotransferase or aspartate aminotransferase at screening.
22. Subjects with a ≧0.3× upper limit of normal (ULN) of gamma glutamyl transferase or alkaline phosphatase at screening.
23. subjects with a ≧2× upper limit of normal (MIN) of direct bilirubin at screening.
24. Subjects with a history of cirrhosis (Child-Pugh Score B or C).
25. Subjects with any history of malignancy in the last year, prior to screening, excluding basal cell carcinoma.
26. Subjects who have used any other investigational drugs within 3 months prior to screening.
27. Use of moderate or strong inhibitors of cytochrome P450 (CYP) 3A4 (eg, ciprofloxacin) within 2 weeks prior to Baseline visit.
28. Use of strong inducers of CYP 3A4 within 2 weeks prior to Baseline visit.
29. Women who are pregnant or nursing at the time of screening, or who intend to be during the study period.
30. Women of child-bearing potential who are not willing to practice an acceptable method of birth control during the study and until 10 days after the last dose of treatment is administered. Hormonal contraceptives as the sole method of birth control are not acceptable. Acceptable (non-hormonal) methods of birth control include surgical sterilization, intrauterine devices, partner's vasectomy (with appropriate post-vasectomy documentation of absence of sperm in ejaculate), or barrier methods (condom or diaphragm with spermicide), Hormonal methods of birth control are permitted but must be supplemented with one of the non-hormonal methods listed above.
31. A known drug hypersensitivity that would preclude administration of the study drug, such as hypersensitivity to: mannitol, meglumine or sodium stearyl fumarate.
32. Subjects unable to comply with the planned schedule of study visits and study procedures.
33. Subjects who are participating in another clinical study.

Withdrawal Criteria/Treatment Failure
1. A subject may be withdrawn from the study in the event of intercurrent illness, AEs, pregnancy, or other reasons concerning the health or well-being of the patient, or in the event of lack of cooperation.

Monitoring Plan and Safety Stopping Rules

In any of the events listed below, the subject's participation in the study is discontinued immediately. The subject is followed until resolution or stabilization of symptoms or lab abnormalities:
1. Any increase in ALT or AST to 3 times ULN, combined with either ≧1.5 times ULN elevation of INR or ≧2 times ULN elevation of total bilirubin.
2. Any increase in ALT or AST ≧3 times ULN, with the appearance of worsening of nausea, vomiting, fever, rash, or eosinophilia.
3. Any increase in ALT or AST to levels ≧5 but <8 times ULN, which is persistent for 2 weeks of repeated measurements.
4. Any increase in ALT or AST to levels 8 times ULN.
5. In any case where monitoring of liver enzymes cannot be performed according to the protocol guidance.

Outcome Measures

Clinical Effect

Primary Efficacy Measure and Endpoint
The primary efficacy endpoint is the proportion of patients achieving Remission at Week 12.

Secondary Efficacy Measures and Endpoints
Proportion of patients achieving Response 100 at Week 12.
2. CDEIS mean change from Baseline to Week 12.
3. Proportion of patients achieving (a) a score of ≦1 on the 11 point NPS (averaged over 7 consecutive days), and (b) an average frequency of 55$ Type 6 (fluffy pieces with ragged edges, a mushy stool) or Type 7 stools (watery, no solid pieces—entirely liquid)<1˜5/day (averaged aver 7 consecutive days) at Week 12.

Other Efficacy Measures and Endpoints
1. Proportion of patients achieving Remission, Response 70, and Response 100 at each visit.
2. CDAI mean change from. Baseline at each visit.
3. Time to Remission,
4. Time to Response 70.
5. Time to Response 100.
5. Proportion of patients achieving CDEIS response at Week 12.
7. Proportion of patients achieving CDEIS improvement at Week 12.
8. Proportion of patients achieving CDEIS remission at Week 12.
9. Proportion of patients achieving mucosal healing at Week 12.
10. Proportion of patients achieving at least a 50% reduction compared to Baseline in the surface ulceration at Week 12 (using the same 5 intestinal segments used to derive the CDEIS, each rated on a scale of 0 to 10, such that the maximum possible score is 50 paints).
11. Proportion of patients achieving fecal calprotectin response by visit.
12. Fecal calprotectin mean percentage change from Baseline by visit.
13. Proportion of patients achieving CRP response by visit.
14. CRP mean percentage change from Baseline by visit.
15. Mean change from Baseline to Week 12 in total IBDQ score.
16. Proportion of patients with an IBDQ response at Week 4 and Week 12.
17. Proportion of patients with IBDQ remission at Week 4 and Week 12.
18. Mean change in scores from Baseline at Week 4 and Week 12 for each of the 4 IBDQ domain scores.
19. Proportion of patients who achieve a change in IBDQ domain which is larger than the Minimum Important Difference (MID) for that domain at Week 4 and at Week 12. (MID for the 4 domain: Bowel symptoms=5, Emotional symptoms-6, Social function=3 and Systemic symptoms=3).
20. Mean change from Baseline to Week 0.4 and Week 12 in each of the 4 domains of the Crohn's Disease Specific Work Productivity and Activity Impairment CD-specific (WPAI:CD) questionnaire.
21. Far each of the 4 domains of WPAI:CD proportion of patients with change from Baseline to Week 4 and Week 12 which is ≧7% (7% is considered HID).
22. Mean change of EQ-5D visual analog scale (VAS) scores from Baseline to Week 4 and Week 12.
23. Percentage of patients reporting each level in each EQ 5D dimension at Week 4 and Week 12.
24. Correlation between CDAI Remission and CDEIS improvement at Week 12.
25. Correlation between change from Baseline to Week 12 in CDAI score and the change from Baseline to Week 12 in CDEIS score.
26. Correlation between change from Baseline to Week 12 in CDEIS score and the change from Baseline to Week 12 in fecal calprotectin level.

Safety/Tolerability
1. Adverse events (AEs).
2. Clinical laboratory values.
3, Vital signs.
4. ECG.
5. Proportion of subjects who prematurely discontinue treatment.
6. Proportion of subjects who prematurely discontinue treatment due to AEs.
7. Time to premature treatment discontinuation.
8. Time to premature treatment discontinuation due to AEs.

Pharmacokinetics/Population PK

Blood samples for PPK evaluation is collected at Weeks 4 and e from all patients.

Results

0.25 mg/day

Administration of 0.25 mg/day oral dose of laquinimod to subjects suffering from active moderate to severe Crohn's disease is effective to reduce at least a symptom of Crohn's disease in the subject, induce clinical response, induce and/or maintain clinical remission, and/or inhibit, disease progression and/or disease complication in the subject. Specifically, the administration of the laquinimod as described herein is effective to reduce the subject's Crohn's Disease Activity index score, induce and/or maintain CDEIS improvement and/or remission, induce mucosal healing, induce and/or maintain at least a 50% reduction compared to baseline in surface ulceration at Week 12, lower the subject's C-Reactive Protein level and/or fecal calprotein level, induce IBDQ response, induce and/or maintain IBDQ remission, and improvement in WPAI:CD questionnaire and EQ-50 visual analog scale (VAS) scores.

As compared to 0.5 mg/day, administration of 0.25 mg/day oral dose of laquinimod to subjects suffering from active moderate to severe Crohn's disease is at least as effective, or more effective, to reduce at least a symptom of Crohn's disease in the subject, induce clinical response, induce and/or maintain clinical remission, and/or inhibit disease progression and/or disease complication in the subject. Specifically, the administration of the laquinimod as described herein is at least as effective as 0.5 mg/day to reduce the subject's Crohn's Disease Activity Index score, induce and/or maintain CDEIS improvement and/or remission, induce mucosal healing, induce and/or maintain at least a 50% reduction compared to baseline in surface ulceration at Week 12, lower the subject's C-Reactive Protein level and/or fecal calprotein level, induce IEDQ response, induce and/or maintain IBDQ remission, and improvement in WPAI:CD questionnaire and EQ-50 visual analog scale (VAS) scores, while also maintaining or reducing adverse effects.

### Example 2

### Clinical Trial (Phase III)—Maintenance of Remission Study of Laquinimod in Crohn's Disease Patients

A phase III, randomized, double-blind, placebo-controlled 52-week maintenance of remission study of laquinimod is conducted in Crohn's disease patients.

Study Title

A Phase III, Multicenter, Randomized, Double-Blind, Placebo-Controlled Study to Evaluate the Efficacy, Safety, Tolerability of Laquinimod in Active Moderate to Severe Crohn's disease.

Number of Investigational Centers

The number of investigational centers is the amount of centers that participated in I of 3 induction studies (TV5600-IMM-30009, TV5600-IMM-30012, and TV5600-IMM-30013).

Number of Subjects

The total number of patients enrolled in the study is determined by the proportion of patients included in the primary efficacy cohort (PBC). The PEC consists only of patients who, in 1 of 3 induction studies (TV5500-IMM-30009, TV5600-IMM-30012, and TV5600-1M-30011), were treated with an effective laquinimod dose (based on the overall benefit/risk results of study TV15600-IMM-30009). The PEC includes 150 patients per arm. The total number of patients enrolled in the study is greater than the size of the PEC, as some of the patients had been treated with placebo or an ineffective laquinimod dose in the induction studies.

Investigational Medicinal Product (IMP) Dosage

One or more capsules containing laquinimod 0.25 mg, laquinimod 0.5 mg, or matching placebo are administered orally once daily (QD)

1^{st} arm—laquinimod 0.25 mg (1×0.25) and placebo capsule for 0.5 mg (1× placebo capsule for 0.5 mg);

2^{nd} arm—laquinimod 0.5 mg 41×0.5) and placebo capsule for 0.25 mg (1× placebo capsule for 0.25 mg); and

3^{rd} arm—placebo capsule for 0.25 mg (1× placebo capsule for 0.25 mg) and placebo capsule for 0.5 mg (1× placebo capsule for 0.5 mg),

The 0.25 mg and 0.5 mg laquinimod capsules are packaged in high density polyethylene, 50-mL bottles with 2 g of silica gel in cap.

During the study period the CDAI score is assessed. Patients axe required to report daily symptoms diary data for the 7 days prior to each visit of the treatment period. The scores obtained from the 7 day diaries completed prior to the Baselines visit (taken from the diaries from their final week in the previous induction study) and to each of the post Baseline visits (starting at Week 6) contribute to a total CDAI score at each of these time points. Assessments or abdominal pain (using the NPS) and watery stool frequency (using the BSS) is also be performed at Baseline and at Weeks 5, 12, 24, 36, and as with CDAI, NPS/BSS data was recorded doily for 7 days prior to each visit.

Allowed previous standard of care treatment is kept stable throughout the study (including the follow-up period, as defined herein).

Study Duration

Each a (Dose Group) is Evaluated for Up to 53 Weeks

Treatment period: Up to 53 weeks

Follow-up period: 4 weeks

Study Population

Patients with a Response at Week 12 in any of the induction studies, TV5500-IMM-30009, TV5600-IMM-30012, or TV5500-IMM-10013, are re-randomized into this placebo-controlled maintenance study. Response at Week 12 in those induction studies is defined as a Crohn's Disease Activity Index (CDAI) reduction of at least 10 points from Baseline at Week 12. No new patients are included in this maintenance study.

Study Assign

This is a phase III, randomized, double-blind, placebo-controlled 52-week maintenance of remission study in patients with CD who had a response in a previous induction study.

This study investigates laquinimod doses at 0.25 mg and 0.5 mg daily. Each dose is studied in a distinct arm.

Subjects are randomized in a 1:1:1 ratio for one of the following treatment arms:

I. Laquinimod 0.25 mg.

2. Laquinimod 0.5 mg.

3. Placebo.

The laquinimod capsules (0.25 mg and 0.5 mg) are of identical appearance to their matching placebo (white opaque cap and body; hard gelatin capsules filled with white to off-white granulate) to maintain study blinding.

Scheduled in-clinic visits are conducted at Baseline (Visit 1, Week 0) and at Weeks 6, 12, 24, 36, and 52. The Baseline visit of this study is the same as the final Week 12b visit in the previous induction study, with some procedures taken from Week 12a.

Unscheduled visits for safety or for any other reason may be conducted at any time during the study. Patients have a follow-up period of 4 weeks.

During the study period the CDAI score is assessed in addition to routine safety laboratory tests and PK analysis.

Patients are required to report daily symptoms diary data for the assessment of the CDAI, BSS and UPS from the Baseline visit until the end of their participation in the study at Follow-up. The scores obtained from the 7 consecutive days of CDAI data completed prior to the baseline visit (taken from the diaries from the Week 12a Visit in the previous induction study) and for 7 consecutive days prior to each subsequent visit contribute to a total CDAI score at each time point. Evaluation of abdominal pain (using the NPS) and stool frequency and consistency (using the MS) is based on the average of the values 7 days prior to the Baseline visit (taken from the diaries from the Week 12a Visit in the previous induction study) and for 7 consecutive days prior to each subsequent visit.

Ileonclonoscopy is performed at Baseline Week 12b Visit from the induction study) and Week 52 to directly assess endoscopic disease and mucosal healing. The timing of the ileocolonoscopy is arranged so that it takes place after the final day on which data is recorded for the Week 52 CDAI assessment, and allows time for adequate ileocolonoscopy preparation; consequently, the Week 52 ileocolcnoscopy is performed separately from but within 7 days of) the other Week 52 assessments each endosoopy is recorded, read, and scored by a central reader for an endoscopic score of cDEIS, and mucosal healing determination,

In addition, patients are required to complete the IBDQ, CD specific WPAI (WPAI:CD), and EQ-50 questionnaires at baseline and Weeks 12, 24, 36, and 52. Fecal calprotectin is assessed from stool samples collected at baseline and Weeks 6, 12, 24, 36, and 52. Serum CRP levels are assessed as part of the standard clinical laboratory evaluations.

Allowed previous standard of care treatment is kept stable throughout the study (including the follow-up period as defined herein).

PK lima lye s

Population PK Study (PPK)

Blood samples for PPK evaluation are collected at Weeks 6, 24, and 52 from all subjects in all arms.

Allowed Concomitant Medications During Study

In general the dose of allowed concomitant medication is kept stable throughout the study. Any new medication/treatment for CD or dose increase not allowed by the protocol, CD surgery, results in early treatment discontinuation. Decrease in dose or dose regiment, not allowed by the protocol, also results in major protocol violation. Failure to taper down corticosteroids as required by the protocol may be considered TF for efficacy analysis.

All concomitant medications that the patient was taking at study initiation and changes or new medications during the study are to be recorded on the concomitant medications log,

The use of 5-ASA compounds is kept stable throughout the study.

Antibiotics

The use of antibiotics for the treatment of Cretin's disease is kept stable throughout the trial. Managing acute infections (not related to Crohn's disease) is allowed. The use of antibiotics, such as ciprofloxacint erythromycin, clarithromycin, troleandomycin and telithromycin which inhibit CYP3A4, is not allowed.

Glucocorticosteroids

All patients on oral steroids (up to 20 mg prednisolone [or equivalent] or 6 mg budesonide) at study entry are required to taper their steroid dose until steroid free on Week 12. For the purposes of this study, patients are considered steroid free after the following taper scheme: prednisolone (or equivalent)—in decrements not greater than 5 mg/2 weeks for tapering down to 10 mg/day, and in decrements not greater than 2.5 mg/2 weeks for tapering down to lower than 10 mg/day until at 5 mg/day or off; budesonide, 3 mg/4 weeks until off by Week 12. From Week 12 and until the and of the study (52 weeks)—steroid dose is to remain stable. Failure to taper down corticosteroids as required by the protocol is considered TF for efficacy analysis.

Immunosuppressants

Immunosuppressive treatment allowed by the protocol (AZT/6-MP/MTX) is kept stable throughout the study. Addition of new immunosuppressive drugs is not allowed.

Other
1. Antidiarrehal drugs, analgesics, NSAIDs and topical preparations are allowed (including topical dermatological, ophthalmological or inhaled steroids), on the discretion of the investigator.
2. The use of probiotics and probiotics is kept stable throughout the study.

Inclusion/Exclusion Criteria

Inclusion Criteria

Subjects must meet all the inclusion criteria to be eligible:
1. Subjects completed Week 12 of the previous induction study (TV5600-IMM-30009, TV5600-114M-30012, or TV5600-IMM-30013).
2. Subjects have a clinical Response. (defined as a CDAI reduction of at least 70 points from baseline at Week 12 of the induction study) from previous induction studies (TV5600-IMM-30009, TV5600-IMM-30012, or TV5600-IMM-30013).
3. Subjects willing and able to provide written, informed consent.

Exclusion Criteria

Any of the following excludes the subject from entering the study:
1. Subjects who, in the opinion of the investigator, had a finding in the previous induction study (e.g., AE, clinical laboratory finding) that would make them unsuitable for inclusion in this maintenance study, taking into account the inclusion and exclusion criteria of the induction study,
2. Subjects unable to comply with the planned schedule of study visits and study procedures (including patient reported assessments).
3. Women of child-bearing potential who are not willing to practice an acceptable method of birth control during the study and until 30 days after the last dose of treatment is administered (Acceptable methods of birth control in this study are: surgical sterilization, intrauterine devices, oral contraceptive, contraceptive patch, long-acting injectable contraceptive, partner's vasectomy, a double-protection method (condom or diaphragm with spermicide)).
4. Women who are pregnant or nursing, or who intend to be during the study period.
5. Subjects who are participating in another clinical study.

Withdrawal Criteria/Treatment Failure
1. A subject may be withdrawn from the study in the event of intercurrent illness, AEs, pregnancy, or other reasons concerning the health or well-being of the patient, or in the event of lack of cooperation.

Monitoring Plan and Safety Stopping Rules

In any of the events listed below, the subject's participation in the study is discontinued immediately. The subject is followed until resolution or stabilization of symptoms or lab abnormalities:
1. Any increase in ALT or AST to ≧3 times ULN, combined with either ≧1.5 times ULN elevation of TER or ≧2 times ULN elevation of total bilirubin.
2. Any increase in ALT or AST to ≧3 times ULN, with the appearance of worsening of nausea, vomiting, fever, rash, or eosinophilia.
3. Any increase in ALT or AST to levels ≧5 but <8 times ULN, which is persistent for 42 weeks of repeated measurements,
4. Any increase in ALT or AST to levels <8 times ULN.
5. In any case where monitoring of liver enzymes cannot be performed according to the protocol guidance.

Outcome Measures

Clinical Effect

Primary Efficacy Measure and Endpoint
1. The primary efficacy endpoint is the proportion of patients achieving Remission at Week 52.

Secondary Efficacy Measures and Endpoints
1. Proportion of patients achieving CDEIS Improvement at Week 52.
2. Proportion of patients in sustained Remission.
3. Proportion of patients in Steroid Free Remission at Week 52 (evaluated only for patients who were on steroids at Baseline).
4. Proportion of patients achieving (a) a score of ≧1 on the 11 point NPS (averaged over 7 days), and (b) an average frequency of BSS Type 7 stools (watery, no solid pieces entirely liquid) (1.5/day (averaged over 7 days) at Week 52.

Other Efficacy Measures and Endpoints
1. Proportion of patients achieving Remission at each visit.
2. CDAI mean change from baseline at each visit.
3. Proportion of patients achieving CDEIS Response at Week 52.
4. Proportion of patients achieving CDEIS Remission at Week 52.
5. CDEIS mean changes from baseline to Week 52.
6. Proportion of patients achieving mucosal healing at Week 52.
7. Fecal calprotectin mean percent change from baseline by visit.
8. CRP mean percent change from Baseline by visit.
9. Mean total IBDQ score change between baseline and Weeks 12, 24, 35, and 52.
10. Proportion of IBDQ Remission at Weeks 12, 24, 36, and 52.
11. Mean change from baseline to Weeks 12, 24, 35, and 52 in IBDQ score for each of the 4 IBDQ domains.
12. Mean change from baseline to Weeks 12, 24, 36, and 52 in each of the 4 domains of the CD specific WPAI (WPAI:CD) questionnaire.
13. Mean change of EQ-5D visual analog scale scores from Baseline to Weeks 12, 24, 36, and 52.
14. Percentage of patients reporting each level in each EQ 5D dimension at Weeks 12, 24, 36, and 52.
15. Correlation between Remission and CDEIS Improvement et Week 52.
16. Correlation between change from baseline to Week 52 in CDAI scare and change from baseline to Week 52 in CDEIS score.
17. Correlation between change from baseline to Week 52 in fecal calprotectin level and change from baseline to Week 52 in CDEIS score.

Safety/Tolerability
1. Adverse events (AES).
2. Clinical laboratory values.
3. Vital signs.
4. ECG.
5. Proportion of subjects who prematurely discontinue treatment.
6. Proportion of subjects who prematurely discontinue treatment due to AEs.
7. Time to premature treatment discontinuation.
8. Time to premature treatment discontinuation due to AEs,

Results

0.25 mg/day

Administration of 0.25 mg/day oral dose of laquinimod to subjects suffering from active moderate to severe Crohn's disease is affective to reduce at least a symptom of Crohn's disease in the subject, induce clinical response, induce and/or maintain clinical remission, and/or inhibit disease progression and/Or disease complication in the subject. Specifically, the administration of the laquinimod as described herein is effective to reduce the subject's Crohn's Disease Activity Index score, induce and/or maintain CDEIS improvement and/or remission, induce mucosal healing, induce and/or maintain at least a 50% reduction compared to baseline in surface ulceration at Week 12, lower the subject's C-Reactive Protein level and/or fecal calprotein level, induce IBDQ response, induce and/or maintain IBDQ remission, and improvement in WPAI:CD questionnaire and EQ-5D visual analog scale (VAS) scores.

As compared to 0.5 mg/day, administration of 0.25 mg/day oral dose of laquinimod to subjects suffering from active moderate to severe Crohn's disease is at least as effective, or more effective, to reduce at least a symptom of Crohn's disease in the subject, induce clinical response, induce and/or maintain clinical remission, and/or inhibit disease progression and/or disease complication in the subject. Specifically, the administration of the laquinimod as described herein is at least as effective as 0.5 mg/day to reduce the subject's Crohn's Disease Activity Index score, induce and/or maintain CDEIS improvement and/or remission, induce mucosal healing, induce and/or maintain at least a 50% reduction compared to baseline in surface ulceration at Week 12, lower the subject's C-Reactive Protein level and/or fecal calprotein level, induce IBDQ response, induce and/or maintain IBDQ remission, and improvement in WPAI:CD questionnaire and EQ-5D visual analog scale (VAS) scores, while also maintaining or reducing adverse effects.

### Example 3

### Clinical Trial Clinical Trial (Phase III)—Induction and Maintenance Stud of Laquinimod in Crohn's Disease Patients

A phase III, randomized, open-label 64-week induction and maintenance study of laquinimod is conducted in Crohn's disease patients,

Study Title

A Phase III, Open-Label Induction and Maintenance Study in Active Moderate to severe Crohn's Disease Patients.

Number of Investigational Canters

The number of investigational centers is the amount of centers that participated in 1 of 2 induction studies (TV5600-IMM-30009 or TV5600-IMM-30012).

Number of Subjects

All patients who did not show Response to either laquinimod or placebo in either of induction studies TV5600-IMM-30009 or TV5600-IMM-30012 are invited to participate and enroll into this open label induction and maintenance study. It is planned that studies TV5600-IMM-30009 and TV5600-IMM-30012 would recruit approximately 540 and 380 patients, respectively.

Investigational Medicinal Product (IMP) Dosage

One capsule containing laquinimod 0.25 mg or laquinimod 0.5 mg is administered orally once daily at the same hour:

1^{st} arm—laquinimod 0.25 mg (1×0.25); and

2^{nd} arm—laquinimod 0.5 mg (1×0.5).

The 0.25 mg and 0.5 mg laquinimod capsules are packaged in high density polyethylene, 50-ml, bottles with 0.2 g of silica gel in cap.

Study Duration

Each arm (dose group) is evaluated for up to 64 weeks

Treatment period: Up to 64 weeks

Follow-up period: 4 weeks

Study Population

Patients who did not demonstrate a Response during either of the 12 week induction studies TV5600-1M-30009 or TV5600-IMM-30012. Response at Week 12 in those induction studies is defined as a Crohn's Disease Activity Index (COAT) reduction, of at least 70 points from Baseline. It is anticipated that many non-responders received placebo in the induction studies, so that TV5600-IMM-30011 is their first opportunity to be exposed to any dose of laquinimod. No new patients are included in this study.

Study Design

This is a Phase III, open-label induction and maintenance study in patients with active moderate to severe CD who did not demonstrate a clinical response in a pivotal induction study (TV5600 IMM 30009 or TV5600 IMM 30012).

This study investigates laquinimod doses at 0.25 mg and 0.5 mg daily. Each dose is studied in a distinct arm.

Subjects are randomized in a 1:1 ratio for one of the following treatment arms:

1. Laquinimod 0.25

2. Laquinimod 0.5 mg.

The laquinimod capsules are packaged in round 50-ml, white high density polyethylene bottles (35 capsules per bottle).

Treatment duration is up to 64 weeks. The initial phase of the treatment duration is 12 weeks. Scheduled in-clinic visits are conducted at Baseline (Week 0, Visit 1) and at Weeks 2, 4, 8, and 12. The Baseline visit (Week 0, Visit 1) for this study is the same as the Week 12 visit in the relevant induction study (i.e., not an additional visit). Some additional procedures may be performed on this day, as a part of the TV5600-114M-30011 protocol but not the induction study protocol. Unscheduled visits for safety or for any other reason are conducted at any time during the study. At Week 12 (and at subsequent visits), patients undergo a clinical assessment and are offered either release from the study or continuation of maintenance therapy at the existing dose. For patients who choose to continue maintenance therapy after Week 12, treatment and assessments are performed at Week 16 and then every 2 months until Week 64 (i.e., Weeks 24, 32, 40, 48, 56, and 64). All patients also have an assessment at Follow-up, 4 weeks following completion of the study (Week 68), upon release from the study, or early termination.

During the study period, routine safety AEs, clinical laboratory values, vital signs, and electrocardiogram [ECG] are assessed. Fecal calprotectin is assessed from stool samples collected at all visits up to Week 64/Early Termination. Serum CRP levels are assessed as part of the standard clinical laboratory evaluations.

Patients are required to report daily symptoms diary data for the assessment of the CDAI, BSS, and NPS from Week 0 (the last visit in the prior induction study) until the end of their participation in this study at Follow-up. The MAI scores obtained from the 7 consecutive days prior to the Baseline visit (taken from the diaries from Week 12 in the previous induction study) and for 7 consecutive days prior to each subsequent visit contribute to a total COAL score at each time point.

Evaluation of abdominal pain (using the NPS) and Type 6 or Type 7 stool frequency and consistency (using the BSS) are based on the average of the values 7 days prior to Baseline and each study visit.

Patients are required to complete the IBDQ, WPAI:CD, and EQ-50 questionnaires at Weeks 4 and 12 during the induction phase and at Weeks 16, 32, 48, and 64 during the maintenance phase.

No forced corticosteroid taper is required for maintenance. Any new medication/treatment for CD or dose increase not allowed by the protocol, CD surgery, biologic treatment, or new immunosuppressive drugs, throughout the study treatment period, results in treatment discontinuation.

Allowed Concomitant Medications During Study

Any new medication/treatment for CD or dose increase not allowed by the protocol, CD surgery, biologic treatment, or new immunosuppressive drugs, throughout the study treatment period, results in early treatment discontinuation.

All concomitant medications that the patient was taking at study initiation and changes or new medications during the study is recorded on the concomitant medications log.

5-ASA Compounds

The use of 5-ASA compounds is kept stable throughout the study.

Antibiotics

The use of antibiotics for the treatment of Cretin's disease is kept stable throughout the trial. Managing acute infections (not related to Crohn's disease) is allowed. The use of antibiotics, such as erythromycin, clarithromycin, troleandomycin and telithromycin which inhibit CYP3A4, is not allowed.

Glucocorticosteroids

No forced corticosteroid taper is required for maintenance.

Intravenous or intramuscular GCS doses or GCS enemas are not be allowed.

Immunosuppressants

Immunosuppressive treatment allowed by the protocol (AZT/6-MP/MTX) is kept stable throughout the study. Addition of new immunosuppressive drugs is not allowed.

Other
1. Antidiarrehal drugs, analgesics, NSAIDs and topical preparations are allowed (including topical dermatological, ophthalmological or inhaled steroids), at the discretion of the investigator.
2. The use of prebiotics and probiotics is kept stable throughout the study.

Inclusion/Exclusion Criteria

Inclusion Criteria

Subjects must meet all the inclusion criteria to be eligible:
1. Subjects completed Week 12 of the previous induction study (TV5600-IMM-30009 or TV5600-IMM-30012).
2. Subjects did not have a clinical Response (defined as a COAT reduction of at least 70 points from baseline at Week 12 of the induction study) from previous induction studies (TV5600-IMM-30009 or TV5600-IMM-30012) to either laquinimod or placebo.
3. Subjects willing and able to provide written, informed consent.

Exclusion Criteria

Any of the following excludes the subject from entering the study:
1. Subjects who, in the opinion of the investigator, had a finding in the previous induction study (e.g., AE, clinical laboratory finding) that would make them unsuitable for inclusion in this maintenance study, taking into account the inclusion and exclusion criteria of the induction study.
2. Subjects unable to comply with the planned schedule of study visits and study procedures (including patient reported assessments).
3. Women of child-bearing potential who were not willing to practice an acceptable method of birth control during the study and until 30 days after the last dose of treatment was administered. Hormonal contraceptives as the sole method of birth control was not acceptable. Acceptable (non-hormonal) methods of birth control include surgical sterilization, intrauterine devices, partner's vasectomy (with appropriate past-vasectomy documentation of absence of sperm in ejaculate), or barrier methods (condom or diaphragm with spermicide). Hormonal methods of birth control were permitted but must have been supplemented with one of the non-hormonal methods listed above.
4. Women who are pregnant or nursing, or who intend to be during the study period.
5. Subjects who are participating in another clinical study.

Withdrawal Criteria/Treatment Failure
1. Each patient was free to withdraw from the study at any time.
2. The investigator also had the right to withdraw a patient from the study in the event of intercurrent illness, AEs, pregnancy, or other reasons concerning the health or well-being of the patient, or in the event of lack of cooperation.

Monitoring Plan and Safety Stopping Rules

In any of the events listed below, the subject's participation in the study is discontinued immediately. The subject is followed until resolution or stabilization of symptoms or lab abnormalities:
1. Any increase in ALT or AST to ≧3 times ULN, combined with either INR >1.5 or total bilirubin >2 times ULN.
2. Any increase in ALT or AST to ≧3 times ULN, which, was accompanied by new or worsening nausea, vomiting, fever, rash, or eosinophilia.
3. Any increase in ALT or AST to levels ≧5 but <8 times ULN, which is persistent for ≧2 weeks of repeated measurements.
4. Any increase in ALT or AST to levels ≧8 times ULN.
5. In any case where monitoring of liver enzymes cannot be performed according to the protocol guidance.

Outcome Measures

Clinical Effect

Primary Efficacy Measure and Endpoint
1. There was no primary efficacy endpoint in this study.

Exploratory Efficacy Measures and Endpoints
1. Fecal calprotectin mean percentage change from Baseline of the present study by visit.
2. CRP mean percentage change from Baseline of the present study by visit.
3. Mean change from Baseline of the present study to Week 12 in total IBDQ score.
4. Proportion of patients with an IBDQ response at week 4 and Week 12.

5. Proportion of patients with IBDQ remission at Week 4 and Week 12.
6. mean change in scores from Baseline of the present study at Week 4 and Week 12 for each of the 4 IBDQ domain scores.
7. Mean change in each of the domains in the CD specific WPAI (WPAI:CD) questionnaire from Baseline of the present study at Week 4 and Week 12.
8. Mean change of EQ-50 visual analog scale (VAS) scores from Baseline of the present study to Week 4 and Week 12.
9. Percentage of patients reporting each level in each EQ 50 dimension at Week 4 and Week 12.
10. Proportion of patients achieving Remission, Response 70, and Response 100 at all visits.
11. COAX mean change from Baseline of the present study by visit.
12. Proportion of patients achieving (a) a score of 1 on the 11 point NPS (averaged over 7 consecutive days), and (b) an average frequency of BSS Type 6 (fluffy pieces with ragged edges, a mushy stool) or Type 7 stools (watery, no solid pieces—entirely liquid)<1.5/day (averaged over 7 consecutive days) at Week 12.

Safety/Tolerability
1. Adverse events (AEs).
2, Clinical laboratory values.
3. Vital signs.
4. ECG.
5. Proportion of subjects who discontinue treatment during the first 12 weeks.
6. Proportion of subjects who discontinue treatment due to AEs during the first 12 weeks.
7. Time to treatment discontinuation.
8. Time to treatment discontinuation due to AES.

Results

0.25 mg/day

Specifically, the administration of the laquinimod reduces the subject's Crohn's Disease Activity Index score, lowers the subject's C-Reactive Protein level and/or fecal calprotein level, induces IBDQ response, induces and/or maintains IBDQ remission, and improvement in WPAI:CD questionnaire and EQ-5D visual analog scale (VAS) scores.

Administration of 0.25 mg/day oral dose of laquinimod to subjects suffering from active moderate to severe Crohn's disease is effective to reduce at least a symptom of Crohn's disease in the subject, induce clinical response, induce and/or maintain clinical remission, and/or inhibit disease progression and/or disease complication in the subject, Specifically, the administration of the laquinimod as described herein is effective to reduce the subject's Crohn's Disease Activity Index score, lower the subject's C-Reactive Protein level and/or fecal calprotein level, induce IBDQ response, induce and/or maintain IBDQ remission, and improvement in WPAI:CD questionnaire and EQ-50 visual analog scale WAS) scores.

As compared to 0.5 mg/day administration of 0.25 mg/day oral dose of laquinimod to subjects suffering from active moderate to severe Crohn's disease is at least as effective, or mare effective, to reduce at least a symptom of Crohn's disease in the subject, induce clinical response, induce and/or maintain clinical remission, and/or inhibit disease progression and/or disease complication in the subject. Specifically, the administration of the laquinimod as described herein is at least as effective as 0.5 mg/day to reduce the subject's Crohn's Disease Activity Index score, lower the subject's C-Reactive Protein level and/or fecal calprotein level, induce ID % response, induce and/or maintain IBDQ remission, and improvement in WPAI:CD questionnaire and EQ-5D visual analog scale (VAS) scores, while also maintaining or reducing adverse effects.

### REFERENCES

1. U.S. Pat. No. 6,077,851, issued Jun. 20, 2000 (BjÖrk et al.).
2. U.S. Patent Pub. No. 2011/0027219 A1, published Feb. 3, 2011 (Tarcic et al.).
3. U.S. Patent Pub. No. 2013/0203807 A1, published Aug. 8, 2013 (Tarcic et al.).
4. PCT International Application Publication No. WO 2007/047863, published Apr. 26, 2007, international filing date Oct. 18, 2006.
5. PCT International Application Publication No. WO 2007/146248, published Dec. 21, 2007, international filing date Jun. 12, 2007.
6. Ben-Horin et al. (2011) Review article: loss of response to anti-TNF treatments in Crohn's disease. Alimentary Pharmacology & Therapeutics; 33(9):987-995 (2011).
7. Best et al. (1976) Development of a Crohn's Disease Activity Index. Gastroenterology; 70:439.494.
8. Best et al. (1979) Rederived values of the eight coefficients of the Crohn's Disease Activity Index (COAT). Gastroenterology; 77:843-6.
9. Chamouard et. al. Diagnostic value of C-reactive protein for predicting activity. Clin Gastrcenterol hepatol; 4:882-887.
10. Colombel et al. (2004) The safety profile of infliximab in patients with Crohn's disease: the Mayo clinic experience in 500 patients. Gastrelenterol; 126:19-31.
11. Colombel et al. (2007) Adlimumab for maintenance of clinical response and remission in patients with Crohn's disease: the CHARM trial. Gastroenterol; 132(1):52-65.
12. Denis at al. (2007) Assessment of Endoscopic Activity Index and Biological Inflammatory Markers in Clinically Active Crohn's disease with Normal C-reactive Protein Serum Level. Inflamm Bowel Dis; 13:1100-1105.
13. Dignass at al. (2010) “The second European evidence-based consensus on the diagnosis and management of Crohn's disease: Current management” Journal of Crohn's and Colitis 4:28-62.
14. MEA 2007. Points to consider on clinical investigation of medicinal products for the management of Crohn's disease. CPMP/EWP/2284/99 Rev.1.
15, Everett and Hamlin (20114 “Evidence-based Use of Anti-TNFα Therapy in Crohn's Disease” Frontline Gastroenterol. 2(3):144-150.
16. Friedman at al., ads. Harrison's Principles of Internal Medicine. New York: McGraw-Hill professional, 2001:1679-92.
17. Froehlich F (2007) “Fibrostenotic Crohn's disease” Digestion. 76(2):113˜5. Epub 2008 February 7.
18. Ghosh et al (2003) Natalizumab for active Crohn's disease. N. Engl 3 Med; 348:24-32.
19. Guindi M and Riddell, R H (2004) “Indeterminate Colitis” J. Clin. Pathol. 57:1233-1244.
20. Hanauer et al. (2002) Maintenance infliximab for Crohn's disease: the accent I randomised trial. Lancet; 359.
21. Hendrickson at al. (2002) Clinical aspects and pathophysiology of inflammatory bowel disease. Clin Microbiol. Rev; 15:79-94.
22, Hammes and Van Deventer (2003) Inflixmab therapy in Crohn's disease; safety issues. Neth 3 Med; 61:100-104.
23. Kasper et al. (2008) Harrison's principles of internal medicine (17th ed.). New York: McGraw-Hill Medical. Publishing 3D Division. ISBN 978-0-07-146633-9
24. Kozuch and Hanauer (2008) Treatment of inflammatory bowel disease: A review of medical therapy. World J Gastroenteroll 14(3):354-377.
25. Laquinimod Investigator's Brochure (IB), Ed. 4, November 2007. Addendum No. 1. June, 2008, Supplement to Investigator's Brochure, Ed. 4, November, 2007.
26. Loftus et al. (2002) The epidemiology and natural history of Crohn's disease in population-based patient cohorts from North America: a systematic review. Aliment Pharmacol Ther; 16(1):51-60.
27. Mannon (2007) “GAIN for Loss: Adalimumab for Infliximab-Regractory Crohn Disease” Ann. Intern. Med. 146(12):888-890.
28. Peyrin-Biroulet et al. (2010) The natural history of adult Crohn's disease in population-based cohorts. Am J Gastroenterol; 105(2):289-297.
29. Rieder et al. (2011) “Predictors of Fibrostenotic Crohn's Disease” Inflamm Bowel Dis. 17(9):2000-2007.
30. Sandborn et al. (2005) Natalizumab induction and maintenance therapy for Crohn's disease. N Engl u Med. 353(18):1912-25.
31. Sandborn et al. (2002) A review of activity indices and efficacy endpoints for clinical trials of medical therapy in adults with Crohn's disease. Gastroenterology. 512-530.
32. Schreiber et al. (2007) Maintenance therapy with centrolizumab pegol for Crohn's disease. N Engl J Med. 357:239-50.
33. Schreiber et al. (2005) A randomized, placebo-controlled trial of certolizumab pegol (CDP870) for treatment of Crohn's disease. Gastroenterul; 129:807-818.
34. Silverberg et al. (2005) Toward an integrated clinical, molecular and serological classification of inflammatory bowel disease: Report of a Working Party of the 2005 Montreal World Congress of Gastroenterology. Can J Gastroenterol. 2005; 19 Suppl A:5-36.
35. Sipponen et al. (2010) Endoscopic Evaluation of Crohn's Disease Activity: Comparison of the CDEIS and the SES-CD. Inflammatory Bowel Diseases, 16(12):2131-35.
36. Sclem et al. (2005) Correlation of C-reactive protein with clinical, endoscOpic, histologic, and radiographic activity in Inflammatory Bowel disease. Inflamm Bowel Dis. 11(8):707-12.
37. Sweetman et al., editors, Martindale: The complete drug reference. London: Pharmaceutical Press. Electronic version, (Edition 35 [2006]).
38. Targan et al. (2007) Uatalizumab for the treatment of active Crohn's disease: result of the ENCORE Trial. Gastroenterology. 132(5):1672-83.
39. Targan et al. (1977) A short-term study of chimeric monoclonal antibody cA2 to tumor necroseis factor alpha for Crohn's disease. N. Engl Med; 337:1029-35.
40, Thomas et al. (2004) Demyelination during anti-tumor necrosis factor alpha therapy with infliximab for Crohn's disease, Inflamm Bowel Dis; 10:28-31.
41. Van Asache et al. (2005) Progressive multifocal leukoencephalopathy after natalizumab therapy for Crohn's disease. N Engl J. Med 2005; 353:362-8.
42. Vermeire et al. (2003) Autoimmunity associated with anti-tumor necrosis factor alpha treatment in Crohn's disease: a prospective cohort study. Gastroenterol; 125:32-9.
43. Wen and Fiocchi (2004) Inflammatory bowel disease: autoimmune immune-mediated pathogenesis? Clin Develop Immunol; 11:195-204.

That is claimed is:

1. A method of treating a subject suffering from Crohn's disease, the method comprising of periodically administering to the subject an amount of laquinimod or pharmaceutically acceptable salt thereof effective to treat the subject, which amount of laquinimod is less than 0.5 mg/day, wherein the subject is naïve to laquinimod and wherein the administration continues for at least 12 weeks.

2. A method of treating a subject suffering from Crohn's disease, the method comprising of periodically administering to the subject an amount of laquinimod or pharmaceutically acceptable salt thereof effective to treat the subject, which amount of laquinimod is less than 0.5 mg/day and wherein the subject is being treated with another Crohn's disease therapy at baseline.

3. The method of claim 1 or 2, wherein the amount of laquinimod is 0.25 mg/day.

4. The method of any one of claims 1-3, wherein the amount of laquinimod is effective to reduce a symptom of Crohn's disease in the subject.

5. The method of claim 4, wherein the amount of laquinimod is effective to induce or maintain clinical remission in the subject.

6. The method of claim 4, wherein the amount of laquinimod is effective to induce C-Reactive Protein response.

7. The method of claim 4, wherein the amount of laquinimod is effective to reduce an endoscopic disease activity in the subject.

8. The method of claim 7, wherein the endoscopic disease activity is measured by the Crohn's Disease Endoscopic Index of Severity (CDEIS).

9. The method of claim 7, wherein the amount of laquinimod is effective to induce CDEIS response.

10. The method of claim 7, wherein the amount of laquinimod is effective to induce CDEIS improvement.

11. The method of claim 7, wherein the amount of laquinimod is effective to induce CDEIS remission.

12. The method of claim 7, wherein the amount of laquinimod is effective to induce mucosal healing.

13. The method of claim 7, wherein the endoscopic disease activity is measured by the Simple Endoscopic Score for Crohn's Disease (SES-CD).

14. Laquinimod for use in treating a subject suffering from Crohn's disease, wherein the amount of laquinimod is less than 0.5 mg/day, wherein the subject is naïve to laquinimod and wherein the administration continues for at least 12 weeks.

15. Laquinimod for use in treating a subject suffering from Crohn's disease, wherein the amount of laquinimod is less than 0.5 mg/day, wherein the subject is being treated with another Crohn's disease therapy at baseline.

16. A pharmaceutical composition comprising an amount of laquinimod for use in treating a subject suffering from Crohn's disease, wherein the amount of laquinimod is less than 0.5 mg, wherein the subject is naïve to laquinimod and wherein the administration continues for at least 12 weeks.

17. A pharmaceutical composition comprising an amount of laquinimod for use in treating a subject suffering from Crohn's disease, wherein the amount of laquinimod is less than 0.5 mg, wherein the subject is being treated with another Crohn's disease therapy at baseline.

18. Use of an amount of laquinimod or a pharmaceutically acceptable salt thereof in the manufacture of a medicament for the treatment of a subject suffering from Crohn's disease, wherein the amount of laquinimod is less than 0.5 mg, wherein the subject is naïve to laquinimod and wherein the administration continues for at least 12 weeks.

19. Use of an amount of laquinimod or a pharmaceutically acceptable salt thereof in the manufacture of a medicament for the treatment of a subject suffering from Crohn's disease, wherein the amount of laquinimod is less than 0.5 mg, wherein the subject is being treated with another Crohn's disease therapy at baseline.

20. A therapeutic package for dispensing to, or for use in dispensing to, a subject suffering from Crohn's disease and naïve to laquinimod, which comprises:
a) one or more unit doses, each such unit dose comprising an amount of laquinimod, and
b) a finished pharmaceutical container therefor, said container containing said unit dose or unit doses, said container further containing or comprising labeling directing the use of said package in the treatment of said subject for a period of at least 12 weeks.

21. A therapeutic package for dispensing to, or for use in dispensing to, a subject suffering from Crohn's disease and being treated with another Crohn's disease therapy at baseline, which comprises:
a) one or more unit doses, each such unit dose comprising an amount of laquinimod, and
b) a finished pharmaceutical container therefor, said container containing said unit dose or unit doses, said container further containing or comprising labeling directing the use of said package in the treatment of said subject.
